(19) United States
(12) Patent Application Publication
(10) Pub. No.: US 2026/0191753 A1
(43) Pub. Date: Jul. 9, 2026

(54) TOPICAL FORMULATIONS FOR HAIR GROWTH STIMULATION CONTAINING OXIDIZED FORM OF LOW MOLECULAR WEIGHT CARBOXYLIC ACIDS

(71) Applicant: WiQo S.P.A., Milano (MI) (IT)

(72) Inventors: Rossana Castellana, Trieste (IT); Francesco Piani, Trieste (IT)

(73) Assignee: WiQo S.P.A., Milano (MI) (IT)

(21) Appl. No.: 19/129,687

(22) PCT Filed: Nov. 8, 2023
(86) PCT No.: PCT/EP2023/081210

§ 371 Date: May 14, 2025

Related U.S. Application Data
(60) Provisional application No. 63/383,723, filed on Nov. 15, 2022.

(51) Int. Cl. A61K 8/38; A61K 8/22; A61K 8/36; A61K 31/19; A61K 31/327; A61K 33/40; A61P 17/14; A61P 39/06; A61Q 7/00

(52) U.S. Cl. CPC A61K 8/38; A61K 8/22; A61K 8/36; A61K 31/19; A61K 31/327; A61K 33/40; A61P 17/14; A61P 39/06; A61Q 7/00; A61K 2800/522; A61K 2800/592; A61K 2800/74

(57) ABSTRACT

Topical formulations comprising peroxyacids prepared in situ from trichloroacetic acid, dichloroacetic acid, monochloroacetic acid or acetic acid and hydrogen peroxide are useful for promoting hair growth and for preventing hair loss.

### FIELD OF THE INVENTION

The present invention relates to topical formulations comprising peroxyacids, preferably peroxyacids prepared in situ from trichloroacetic acid, dichloroacetic acid, monochloroacetic acid or acetic acid and hydrogen peroxide, for promoting hair growth.

### BACKGROUND OF THE INVENTION

The “hair loss” may refer to a condition in which there is no hair in the skin where hair should normally exist and may refer to a condition where hair of the scalp falls out. Hair loss may depend on different causes, including various dietary habits, environmental influences, genetic causes, hormonal imbalance, mental stress, exposure to air pollution, to radiation therapies and to certain drugs (Dermatol Clin 31 (2013) 67-73).

Hair loss may be classified in different classes as: hereditary androgenic alopecia (baldness), alopecia areata (AA), tinea capitis (due to fungal infection), telogen alopecia, trichotillomania, hair generation disorders, and the like. Lupus hair loss, folliculitis decalvans, lichen planopilaris, and hair loss by burn and trauma can be correctly defined as examples of cicatricial alopecia.

Androgenetic alopecia (AGA) is a genetically predetermined disorder due to excessive response to androgens which affects up to 50% of males and 20% of females. AGA is characterized by progressive loss of terminal hair of the scalp any time after puberty. AGA is related to 5α-reductase type II activity, which converts testosterone into dihydrotestosterone. The transformation of testosterone into its most active metabolite dihydrotestosterone is the cause of the miniaturisation of the bulb and leads to definitive bulb death and irreversible hair loss.

The most important drug for treating AGA is finasteride, a selective inhibitor of the 5α-reductase type II. This therapy however must be continued for all life on a daily base and after discontinuation the hair fall out again. Moreover, finasteride increases the circulating levels of testosterone which is peripherally aromatized to oestrogens, possibly causing in males sexual dysfunctions, decreased libido and gynaecomastia.

AA is a common autoimmune disorder afflicting hair follicle in the active phase of the hair growth cycle (anagen phase). The most common form of AA treatment is the use of corticosteroids (Canadian Family Physician, Vol 66: July 2020; 499-501), commonly administered through local injections, topical ointment application or orally. While drugs can help hair regrowth, they cannot be taken for long periods since serious side effects may occur. It is also possible that when the medication is suspended, hair loss is re-established.

Other topicals drugs include minoxidil, a drug used for the treatment of AA (Drug Design, Development and Therapy 2019:13 2777-2786), immunosuppressants such as tacrolimus (American Academy of Dermatology, Inc 2005. doi:10.1016/j.jaad.2004.05.019) and cyclosporin A (Skin Pharmacol. 1994; 7 (1-2): 101-4) and immunomodulators such as diphenylcyclopropenone (Postepy Dermatol Allergol. 2018 December; 35 (6): 577-581). Photochemotherapy and systemic immunosuppressive therapy can also be used. Recently, a class of drugs called Janus kinase (JAK) inhibitors demonstrated their potential for the treatment of AA and other dermatologic diseases (J Cutan Med Surg. 2019 May/June; 23 (3): 289-297).

Another approach to stimulate the hair growth is the topical application of an irritant such as squaric acid dibutylester (International Journal of Dermatology, Vol, 3, 5, No, 1, January 1996), anthralin (Dermatol Ther. 2017 July; 30 (4). doi:10.1111/dth.12500. Epub 2017 Jun. 9) and trichloroacetic acid (J. Cosmet Dermatol. 2020; 00:1-7; KR 2012136519). The resulting inflammation seems to inhibit the immune system attack against the hair follicles. On the contrary some substances such as hydrogen peroxide suppresses hair growth due to its cytotoxic effects and ability to induce oxidative stress (J.-A. Seo, et al., J. Dermatol. Sci. 66 (1) (2012) 12-19) and downregulating the GSK-3b/β-catenin signaling pathway (J Dermatol Sci. 2018 January; 89 (1): 91-94). Until now, there is no universally proven therapy that induces and maintains remission of AA in all patients.

### DESCRIPTION OF THE INVENTION

The present invention relates to topical formulations containing peroxyacetic or peroxyhalogenated acetic acids or a combination of acetic acid or halogenated acids with a hydrogen peroxide solution in admixture with suitable carriers, vehicles or complementary useful ingredients, for use for preventing hair loss or improving hair growth.

The peroxyacetic or peroxyhalogenated acetic acids are selected from peroxytrichloroacetic acid, peroxydichloroacetic acid, peroxymonochloroacetic acid or peracetic acid, preferably in a range from 2.0 to 0.8% by weight.

The formulations preferably comprise trichloroacetic acid, dichloroacetic acid, monochloroacetic acid or acetic acid in a range comprised between 33.0 and 0.08% weight and 28-30% hydrogen peroxide. The weight ratio of acetic or halogenated acetic acids to hydrogen peroxide 28-30% is between 22.0 and 5.3.

The formulations may be in form of a kit containing separate administration forms for the hydrogen peroxide solution and for the acetic or halogenated acetic acids.

The carriers, vehicles or complementary useful ingredients are preferably selected from glycerol in concentration by weight 2.0-10.0%, water or a 0.9% NaCl water solution added individually in amount comprised between 52-94% by weight, 30% aqueous ammonia in a concentration by weight ranging from 12.0 to 0.03%.

The pH of the formulations is typically from 2 to 3.

The invention also provides a cosmetic aesthetic or therapeutic method of preventing hair loss or improving hair growth comprising the topical application on the skin of a subject in need thereof of an effective amount of the compositions of the invention.

According to a first embodiment of the invention, the compositions are topically administered at T0, and and administration is continued every other day for a total of 12 days of treatment.

According to an alternative embodiment of the invention, the compositions are topically administered at T0, and administration is continued every other day for 8 days of treatment.

The compositions can be used as such or usually diluted, before administration, with demineralized water or physiological 0.9% NaCl/demineralized water. The final concentration may be adjusted to 0.25 to 25% weight/volume, e.g. to 25%, 1.25% and 0.25% weight/volume. Surprisingly, optionally halogenated peroxyacetic acids, in particular trichloroacetic acid, a compound recently studied to promote hair growth, and hydrogen peroxide (known to suppress hair growth) are able to stimulate hair growth to a higher level than the individual components (i.e. trichloroacetic acid or hydrogen peroxide) used alone. The hair growth stimulating effect has been verified in formulations containing trichloroacetic acid, dichloroacetic acid, monochloroacetic acid or acetic acid and 30% hydrogen peroxide in a weight ratio comprised between 22.0 and 5.3. A similar hair growth stimulating effect was also observed when using formulations containing peroxytrichloroacetic acid, peroxydichloroacetic acid, peroxymonochloroacetic acid or peracetic acid in an effective concentration in the final formulation comprised between 0.8% and 2.0% by weight.

A representative example of formulation of the invention contains trichloroacetic acid and hydrogen peroxide in a molar ratio of 15/1. In a preferred formulation, trichloroacetic acid and 30% hydrogen peroxide represent respectively 33% and 1.5% by weight of the final formulation, further comprising glycerol (10.0% by weight), water (43.5% by weight) and 28-30% aqueous ammonia (12% by weight).

Alternatively, the formulations of the invention contain trichloroacetic acid and peroxytrichloroacetic acid or acetic acid and peroxychloroacetic acid in a relative molecular ratio range of 3.6-3.8; preferably the sum of these acids represent 8.4-3.2% by weight of the formulations. The invention is illustrated in more detail in the following examples.

### Example 1

| Compounds | % weight composition |
|---|---|
| glycerol | 2.5 |
| hydrogen peroxide (30%) | 1.3 |
| aq. ammonia (28-30%) | 3.0 |
| Water (*) | 85.0 |
| trichloroacetic acid | 8.3 |

(*) water can be replaced by a 0.9% NaCl aqueous solution

### Comparative Example 2

| Compounds | % weight composition |
|---|---|
| glycerol | 2.5 |
| aq. ammonia (28-30%) | 3.0 |
| Water (*) | 86.3 |
| trichloroacetic acid | 8.3 |

(*) water can be replaced by a 0.9% NaCl aqueous solution

### Comparative Example 3

| Compounds | % weight composition |
|---|---|
| glycerol | 2.5 |
| hydrogen peroxide (30%) | 1.3 |
| aq. ammonia (28-30%) | 3.0 |
| water (*) | 93.3 |

(*) water can be replaced by a 0.9% NaCl aqueous solution

### Example 4

| Compounds | % weight composition |
|---|---|
| glycerol | 2.5 |
| peroxytrichloroacetic acid | 2.0 |
| aq. ammonia (28-30%) | 2.9 |
| water (*) | 86.2 |
| trichloroacetic acid | 6.4 |

(*) water can be replaced by a 0.9% NaCl aqueous solution

### Example 5

| Compounds | % weight composition |
|---|---|
| glycerol | 2.5 |
| hydrogen peroxide (30%) | 1.2 |
| aq. ammonia (28-30%) | 2.9 |
| water (*) | 90.4 |
| acetic acid | 3.0 |

(*) water can be replaced by a 0.9% NaCl aqueous solution

### Example 6

| Compounds | % weight composition |
|---|---|
| glycerol | 2.5 |
| peroxyacetic acid | 0.8 |
| aq. ammonia (28-30%) | 2.9 |
| water (*) | 91.4 |
| acetic acid | 2.4 |

(*) water can be replaced by a 0.9% NaCl aqueous solution

### Example 7

| Compounds | % weight composition |
|---|---|
| glycerol | 10.0 |
| hydrogen peroxide (30%) | 5.0 |
| aq. ammonia (28-30%) | 11.8 |
| water (*) | 40.2 |
| acetic acid | 33.0 |

(*) water can be replaced by a 0.9% NaCl aqueous solution

### Example 8

| Compounds | % weight composition |
|---|---|
| glycerol | 10.00 |
| hydrogen peroxide (30%) | 0.01 |
| aq. ammonia (28-30%) | 0.03 |
| water (*) | 89.88 |
| trichloroacetic acid | 0.08 |

(*) water can be replaced by a 0.9% NaCl aqueous solution

### Example 9

| Compounds | % weight composition |
|---|---|
| glycerol | 10.00 |
| hydrogen peroxide (30%) | 1.5 |
| aq. ammonia (28-30%) | 12.0 |
| water (*) | 43.5 |
| trichloroacetic acid | 33.0 |

(*) water can be replaced by a 0.9% NaCl aqueous solution

### Test Results on Proliferation of Dermal Papilla Cells

### Confirmation of a Higher Effect of the Formulations of Examples 1, 4, 5, 6, 7, 8 and 9 on Proliferation of Hair Follicle in Comparison to Formulations of Comparative Examples 2 and 3

Human hair follicle dermal papilla cells were seeded in a 96-well plate at a density of 4×10⁵ cells/well, and cultured for 16 hours. The culture medium was replaced by a serum-free medium, and the tested formulation was added respectively in a volume of 1.0 μL, 5.0 μL and 50.0 μL, and the cells were cultured for 1 day. 5 μM of minoxidil solution was used as positive control group of hair loss treatment. The cells were harvested and nuclei were isolated by using a nuclear protein extraction kit (Merck, USA). Then, western blotting was performed with respect to β-catenin by using β-catenin antibodies (Cell Signaling Technology, USA).

The higher promoting effect of the formulations of examples 1, 4, 5, 6, 7, 8 and 9 in comparison to the formulations of comparative examples 2 and 3 on the proliferation of hair follicle cells was established. The treatment with the formulations of the invention proved to increase the movement of β-catenin from the cytoplasm to the nucleus of about +15÷20% with respect to the comparative formulations, confirming that the proliferation mechanism of the dermal papilla cells was activated at an higher level with respect to the comparative formulations 2 and 3.

### In Vitro Evaluation of the Stimulation of Hair Growth Using the Formulations of Examples 1, 4, 5, 6, 7, 8 and 9

The advantageous effects of the invention were confirmed by using an in vitro model for hair follicle research (Huelsken, J. et al., Cell 2001, 105, 533-545; Topouzi, Het al., Blackwell Publishing Ltd.: Hoboken, NJ, USA, 2017; Volume 26, pp. 491-496; Lee, L. F.; Chuong, C. M. Building Complex. Tissues: High. Throughput Screening for Molecules Required in Hair Engineering; Nature Publishing Group: Berlin, German, 2009; Volume 129, pp. 815-817; Biomedicines 2021, 9, 435). The treatment of human hair follicle dermal papilla cells with the formulations of examples 1, 4, 5, 6, 7, 8 and 9 significantly increased (+15÷20%) the movement of β-catenin from the cytoplasm to the nucleus in comparison with the reference formulations of examples 2 and 3 containing respectively solely trichloroacetic acid or 30% hydrogen peroxide.

Accordingly, it was confirmed that, due to the increased movement of β-catenin into the nucleus, the proliferation mechanism of the derma I papilla cells was activated at a higher level with respect to the comparative formulations 2 and 3.

Further In Vitro and Ex Vivo Evaluation of the Stimulation of Hair Growth Using the Formulation 9 Diluted ¼ with NaCl 0.9% w/v in Water (THPF250523)

For the evaluation of the characteristic of the topical formulations of the invention the formulation 9 after dilution to ¼ using NaCl 0.9% w/v in water (experimental code THPF250523) was further investigated as such or after progressive dilutions.

### In Vitro Evaluation of the Formulation of Example 9 (THPF250523) on Pseudoderm Using Two Different Protocols of Administration

NHEK (normal human epidermal keratinocytes), HDF (primary dermal fibroblasts) and ORSK (keratinocytes of the outer sheath of hair follicles) cells were used to recreate a 3D organotypic system, taking advantage of their physiology and their ability to develop a layered epithelium; therefore, NHEK cells were placed on a layer of type I collagen mixed with human dermal fibroblasts (HDF) to create a ‘pseudoderm’ that mirrors human anatomy. Subsequently, the pseudodermis was covered with various cell mixtures and matrices, containing basement membrane and extracellular matrix components, outer reticular sheath keratinocytes (ORSK) and mesenchymal cells (HDF). Pseudoderm was treated both with the product as it was (100%) and with decreasing dosages of the product in order to find the lowest effective dose mimicking topical use.

Two different protocols of administration were followed:
in the protocol A the cells were treated at T0, stimulated every other day and evaluated after 4, 8 and 12 days of treatment;
in the protocol B the cells were always treated at T0, stimulated every other day and evaluated after 4 and 8 days; at day 8, treatments were interrupted and cells were maintained until 12 days.

Both protocols were used to study the main biological activities of the formulation 9 based on substances with antioxidant and anti-inflammatory properties. Since the key mechanisms contributing to hair bulb death can be ascribed to the presence of oxidative stress and inflammatory processes, the production of reactive oxygen species (ROS) and the activation of NFKb (nuclear factor kappaB), TNFa (tumor necrosis factor) and the main cytokines involved were analysed. In addition, mitochondrial metabolism and cell growth were analysed, by crystal violet assay, and the key role of the WNT/beta-catenin pathway in generating follicles. Finally, the main markers involved in cell death processes were studied, such as BAX (bcl-2-like protein 4) and caspases (cysteine-aspartase), important proteins involved in triggering and activating apoptotic processes, and cyclin-dependent kinases, important proteins involved in regulating the cell cycle.

### Cell Cultures

Normal human epidermal keratinocytes (NHEK) from neonatal foreskin were purchased from Lonza (Basel, Switzerland) and cultured in keratinocytes basal medium (KBM, Lonza, Basel) containing keratinocyte growth medium-2 (KGM-2, Lonza, Basel, Switzerland) in an incubator at 37° C., 5% CO₂ and 95% humidity [doi:10.4062/biomolther.2012.20.2.171.]. The experiments were conducted at passage 3-6 and 1×10⁶ cells/mL were used to set up the 3D model. Primary dermal fibroblasts (HDF) were purchased from American Type Cell Culture (ATCC, Cansass, USA) and were cultured in Dulbecco's Modifies Egles Medium (DMEM, Merck Life Sciences, Italy) supplemented with 10% foetal bovine serum (FBS, Merck Life Sciences, Italy) and 2 mM L-glutamine supplemented with penicillin/streptomycin (100 IU/100 μg/ml) and maintained in an incubator at 37° C., 5% CO₂ and 95% humidity. For use as feeder cells, confluent HDFs were treated with 4 μg/ml mitomycin C (Acros Organics, supplied by Thermo Fisher) for 24 h and subcultured in 35 mm Petri dishes at a confluence of 3.5×10⁴ cells/well in a final volume of 2 ml [Zarghami, A et al., Fibers and Polymers. 2015; 16, 1201-1212]. ORSK cultures were used on the basis of previously described methodologies [doi:10.1371/journal.pone.0240454]. Briefly, 5-10 hair follicles, with a clearly visible outer sheath, were harvested and incubated for 30 min at 37° C. in 2 ml hair follicle outer sheath keratinocyte (ORSK) medium consisting of DMEM supplemented with 10% FBS, 2 mM L-glutamine, 100 IU/mL penicillin G, 1 mM ascorbyl-2-phosphate, 2 μg/ml adenine, 10 ng/ml epidermal growth factor, 0.4 μg/ml hydrocortisone, 2 nM triiodothyronine, 0.1 nM cholera toxin, 25 μg/mL gentamicin. At the end of 30 minutes, the follicles were rinsed twice with 1 mL of 1× phosphate buffered saline (PBS) containing 100 μg/mL gentamicin and then incubated with 0.5 mL of 0.25% trypsin-EDTA for up to 40 minutes, pipetting and shaking at 10-minute intervals until the sheath was disrupted: this step resulted in a cell suspension composed only of ORSK cells. Subsequently, the cells were seeded on feeder cultures consisting of HDF cells, previously sown in 35 mm Petri dishes, and incubated for 5-6 days, changing the medium every 2 days. For pseudoderm construction, cells were seeded in EpiLife medium supplemented with Human Keratinocyte Growth Supplement (HKGS, Thermo Fisher Scientific, Waltham, Massachusetts, USA) at a density of 1×10⁴ cells/cm².

### Experimental Protocol

NHEK, HDF and ORSK cells were used to recreate a 3D organotypic system, taking advantage of their physiology and their ability to develop a layered epithelium; therefore, NHEK cells were placed on a layer of type I collagen mixed with human dermal fibroblasts (HDF) to create a ‘pseudoderm’ that mirrors human anatomy. Subsequently, the pseudodermis was covered with various cell mixtures and matrices, containing basement membrane and extracellular matrix components, outer reticular sheath keratinocytes (ORSK) and mesenchymal cells (HDF). Pseudoderm was treated both with the product as it was (100%) and with decreasing dosages of the product in order to find the lowest effective dose mimicking topical use. In more detail, the cells were treated at T0, stimulated every other day and evaluated after 4, 8 and 12 days of treatment (PROTOCOL A). In addition, a second treatment protocol was applied (PROTOCOL B) where the cells were always treated at T0, stimulated every other day and evaluated after 4 and 8 days; at day 8, the treatments were discontinued and the cells were maintained until 12 days. Both protocols were used to study the biological activities of THPF250523. Therefore, since the key mechanisms contributing to hair bulb death can be attributed to the presence of oxidative stress and inflammatory processes, the production of reactive oxygen species (ROS) and the activation of NFKb, TNFa and the main cytokines involved were analysed. In addition, mitochondrial metabolism and cell growth were analysed, by crystal violet assay, and the key role of the WNT/beta-catenin pathway in generating follicles. Finally, the main markers involved in cell death processes were studied, such as BAX and caspases, involved in triggering and activating apoptotic processes, and cyclin-dependent kinases, involved in regulating the cell cycle.

### Pseudoderm Construction

Pseudoderm was prepared with type I collagen (Thermo Fisher Scientific, Waltham, Massachusetts, USA) at a final concentration of 4 mg/mL following the protocol [doi:10.1111/j.1365-2133.2004.06184.x]. Initially, type I collagen (prepared in a Hank's buffered saline solution), FBS and HDF cells (passages 2-8) were mixed in an 8:1:1 ratio to make the coating feeder, resulting in a final concentration of 3.2 mg/mL collagen and 2.5×105 cells/mL. Of this mixture, 1.5 mL was poured into a 24-well plate (Corning Costar, Corning, NY, U.S.A.) and allowed to gel at 37° C. After polymerisation, DMEM with 10% FBS was added and the gels were grown under submerged conditions for 5 days, with medium changes every other day. After this period, the gels fully contracted and were used as pseudoderm. The pseudoderm was transferred with forceps into tissue culture wells (pore size 0.4 μm, polycarbonate, Corning Costar). Subsequently, 1×10⁴ ORSK/mL cells 1×10⁶ NHEK/mL cells were mixed with Matrigel™ prepared previously in 1×PBS (ratio 9:1) and seeded over the pseudodermis. The cells were maintained in culture with DMEM supplemented with 10% foetal equine serum (FCS, Merck Life Science, Milan, Italy) at 37° C. until complete polymerisation.

### MTT Test

After treatment, cell viability analysis was performed using a classical technique based on the MTT in vitro toxicology test kit (Merck Life Science, Rome, Italy) [Ruga, S et al., Int. J. Mol. Sci. 2022, 23, 3805], following the manufacturer's instructions. Indeed, at the end of the treatment, the cells were incubated with the 1% MTT dye for 2 h in an incubator at 37° C., 5% CO² and 95% humidity, then the purple formazan crystals were dissolved in an equal volume of MTT solubilisation solution. The absorbance was analysed using a spectrophotometer (Infinite 200 Pro MPlex, Tecan, Männedorf, Switzerland) at 570 nm with correction at 690 nm, and the results were expressed against the control (0% line), which represented the untreated cells. The results represent the percentage of viable cells compared to the control, allowing the assessment of the stimulation safety.

### Production of Reactive Oxygen Species (ROS)

Quantification of superoxide anion release was obtained following a standard protocol based on cytochrome C reduction [Molinari et al., Foods 2021, 10, 1885], and absorbance in culture supernatants was measured at 550 nm using a spectrophotometer (Infinite 200 Pro MPlex, Tecan, Männedorf, Switzerland). Specifically, 100 μL of cytochrome C (Merck, Milan, Italy) was added to all wells, while 100 μL of superoxide dismutase (Merck, Milan, Italy) and 100 μL of cytochrome C were added to the empty wells; the plate was then incubated for 30 min. Subsequently, 100 μL was taken from each well and the absorbance was measured with a spectrophotometer (Infinite 200 Pro MPlex, Tecan, Männedorf, Switzerland) at 550 nm. The O2 rate was expressed as the mean+SD (%) of reduced cytochrome C nanomoles per microgram of protein compared to the control (line 0).

### ELISA Assay for NFKB

To analyse the DNA-binding activity of NFKB, an enzyme-linked immunosorbent assay (ELISA) was performed, following the manufacturer's instructions (Cayman Chemical Company, Ann Arbor, MI, USA). Briefly, nuclear extracts were prepared using a nuclear extraction protocol [Uberti, F et al., J. Ovarian Res. 2017, 10, 61] and NFk-B contained in these extracts was detected with the addition of a specific primary antibody. An HRP-conjugated secondary antibody was added to provide sensitive colorimetry measured by a spectrometer (Infinite 200 Pro MPlex, Tecan, Männedorf, Switzerland) at 450 nm and the concentration was calculated by comparing the results with the standard curve (range 1.6 to 400 ng/ml). Results were expressed as mean+SD (%) versus control (line 0) of five independent experiments performed in triplicate.

### ELISA Assay for TNFα

TNFα concentration was determined using the TNFα ELISA kit (Merck Life Science, Rome, Italy) following the experimental protocol. Colorimetric intensity was measured at 450 nm using a spectrophotometer (Infinite 200 Pro MPlex, Tecan, Männedorf, Switzerland). The data were calculated by generating a calibration curve (range 24.58 μg/ml to 6000 μg/ml) relating the optical density of the sample to the concentration of the standards. Results were expressed as mean+SD (%) versus control (line 0) of five independent experiments performed in triplicate.

### ELISA Assay for Interleukin 1β (IL-1β)

IL-1β was detected using the Human IL-1β (Interleukin 1β) ELISA kit (FineTest, Wuhan, China) according to the manufacturer's instructions. The plate was read immediately at 450 nm with a spectrophotometer (Infinite 200 Pro MPlex, Tecan, Männedorf, Switzerland). A standard curve relating the colour intensity (OD) to the concentration of the standards (31.25 to 2000 μg/mL) was plotted and the results were expressed as mean±SD (%) versus control (line 0) of five independent experiments performed in triplicate.

### Crystal Violet Staining

The treated cells were also analysed by crystal violet staining to study proliferation. At the end of each time-point, cells were fixed with 1% glutaraldehyde for 15 minutes at RT, washed and stained with 100 μl of 0.1% crystal violet for 20 minutes at RT. To obtain the estimated cell number, 100 μl of 10% acetic acid was added and mixed before reading the absorbance at 595 nm using a spectrometer (Infinite 200 Pro MPlex, Tecan, Männedorf, Switzerland). The estimated number of cells was calculated by comparing the results with the control cells (Control TO). Results were expressed as mean±SD (%) compared to the control (line 0) of five independent experiments performed in triplicate.

### Assay for Wnt Activity

Wnt activity was determined using an ELISA kit (Human Wnt-3a ELISA Kit, MyBiosource, San Diego, CA, USA) according to the manufacturer's instructions [doi:10.1016/j.bbrc.2017.04.110.]. The absorbance of the samples was measured at 450 nm with a spectrophotometer (Infinite 200 Pro MPlex, Tecan, Männedorf, Switzerland) and the results were compared to the standard curve (range 1.57 to 100 ng/ml) and expressed as mean±SD (%) normalised to the control value (line 0).

### Assay for BAX Activity

BAX activity was determined using an ELISA kit (Human Bax ELISA Kit, MyBiosource, San Diego, CA, USA) according to the manufacturer's instructions [doi:10.1016/j.bioorg.2021.105035]. The absorbance of the samples was measured at 450 nm with a spectrophotometer (Infinite 200 Pro MPlex, Tecan, Männedorf, Switzerland) and the results were compared to the standard curve (range 0 to 2000 μg/mL) and expressed as mean±SD (%) normalised to the control value (line 0).

### Caspase 3 Assay

Caspase 3 activity was studied in lysates using an ELISA kit (Caspase 3 (Cleaved) Human ELISA Kit, Thermoscientific, Waltham, MA, USA), according to the manufacturer's instructions [doi:10.1038/s41598-020-61871-w.], by reading the absorbance of the sample at 450 nm with a spectrometer (Infinite 200 Pro MPlex, Tecan, Männedorf, Switzerland). The data were obtained by comparison with a standard curve (0.039 to 2.5 ng/ml) and the results were expressed as mean±SD (%) compared to the control value (line 0) of five independent experiments performed in triplicate.

### ELISA for CDK1

The ELISA kit for CDK1 (MyBiosource, San Diego, CA, USA) was used according to the manufacturer's instructions [doi:10.1016/j.stemcr.2015.01.019.]. HOSEpiC cells were lysed with cold 1× Phosphate-Buffered Saline (PBS, Merck Life Science, Rome, Italy), centrifuged at 5000×g for 5 min and 100 μL of each sample was analysed by reading the sample absorbance at 450 nm with a spectrometer (Infinite 200 Pro MPlex, Tecan, Männedorf, Switzerland). The results were obtained by comparing the data with the standard curve (range 0.156 to 10 ng/ml) and were expressed as a percentage (%) compared to the control (line 0) of five independent experiments performed in triplicate.

### Statistical Analysis

Results are expressed as mean±SD of at least 5 biological replicates for each experimental protocol and each replicate was repeated 3 times for each experimental protocol. Statistical comparisons between groups were performed using one-way ANOVA with Bonferroni's post hoc test using GraphPad Prism 5 (GraphPad Software, La Jolla, CA, USA). A p-value of p<0.05 was considered statistically significant. All densitometric analysis data were normalised to control values (defined as 0). All other data from each experimental protocol were normalised to the percentage control values (defined as 0%).

### Results

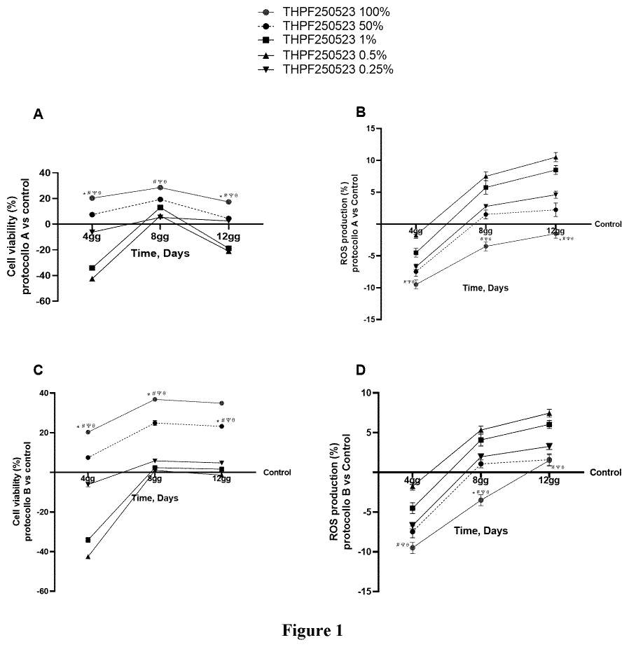

The pseudoderm was treated with two protocols (A and B) for 12 days in order to test the effects of THPF250523, at different concentrations, on cell viability and ROS production. As can be seen from FIG. 1A, following the application of PROTOCOL A, cell viability increased statistically significantly after stimulation with the undiluted test product (100%) compared to the other concentrations tested (p<0.05) throughout the stimulation period. These experiments were also conducted following the application of PROTOCOL B (FIG. 1C), which was shown to improve cell viability, statistically significantly compared to the other concentrations tested (p<0.05), in the interval between day 8 and day 12 of treatment. Furthermore, a better effect was exerted by PROTOCOL B than by PROTOCOL A (p<0.05) as it maintained high cell viability after 8 days of treatment up to 12 days (plateau phase). Both protocols were also evaluated in terms of oxidative stress by analysing the different THPF250523 concentrations after 4, 8 and 12 days of treatment. Indeed, as shown in FIGS. 1B and 1D, ROS production was reduced in a dose-dependent manner (p<0.05), and consistent with cell viability with both treatment protocols. Thus, the data obtained suggest that undiluted THPF250523 has strong potential in maintaining cellular homeostasis without activating oxidative processes.

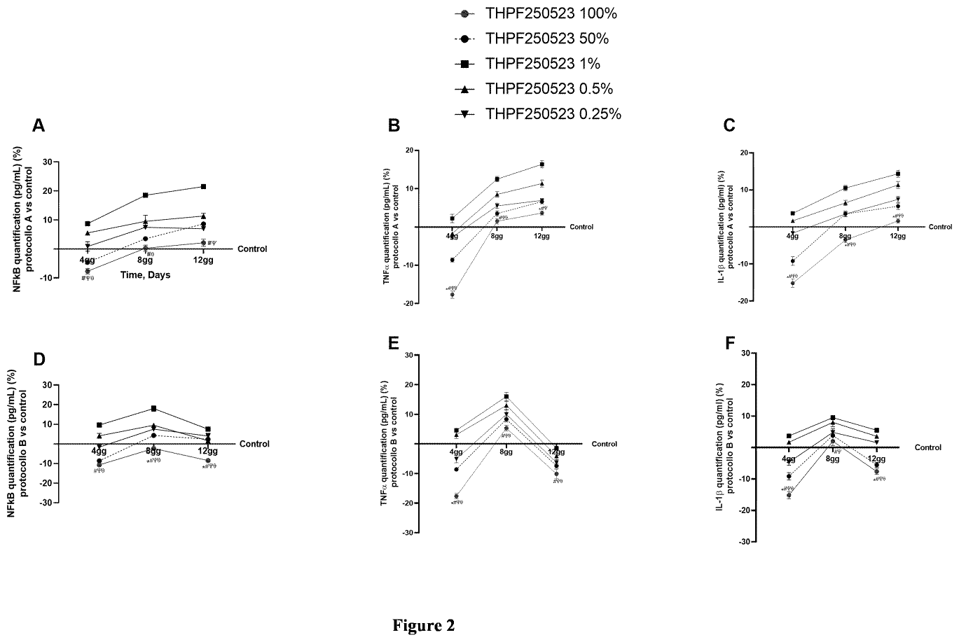

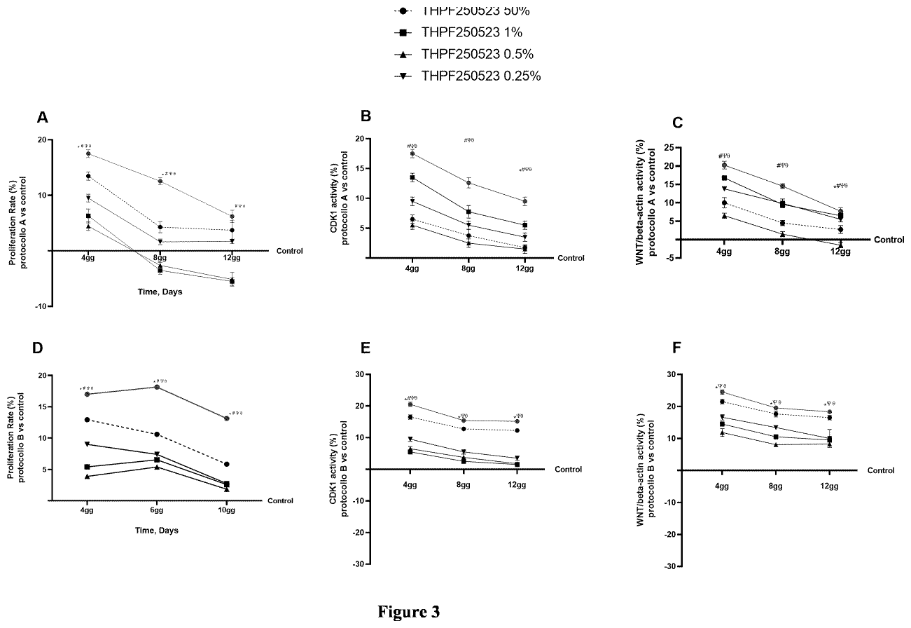

Subsequently, the main biological markers involved in triggering the inflammatory process were investigated on the basis of the results obtained, in order to obtain further confirmation of the positive effects promoted by THPF250523 on hair bulb health. The data shown in FIG. 2 demonstrate that THPF250523 at 100% promotes the maintenance of reduced levels of TNFα, NFkB and IL-1β during the treatment period of PROTOCOL A, compared to the other formulations (p<0.05); in fact, the data demonstrate a good preventive and protective action on the inflammatory mechanism, confirming the results concerning cell vitality and oxidative stress. Consistently, the application of PROTOCOL B (FIG. 3 D-F) also confirmed the previous observations, with statistically significant better results than PROTOCOL A (p<0.05).

The data confirm that THPF250523 maintains the proper functioning of keratinocytes, without inducing significant adverse effects during the 12-day treatment.

Initially, the rate of cell proliferation was analysed by means of crystal violet staining. The highest rate of cell growth was detected following treatment for 12 days with undiluted THPF250523, which was confirmed in both protocols (p<0.05). Specifically, the application of PROTOCOL B showed results consistent with what has been observed previously, emphasising an increase in cell proliferation in the interval between day 8 and day 12, despite treatment discontinuation on day 8 (p<0.05). In addition, the activity of CDK1, a protein included in the class of protein kinases regulating the cell cycle, which is associated with disassembly and reassembly of the nucleus, was investigated. Thus, it is clear that its increased activity indicates increased cell viability and growth. Indeed, the results show that following application of PROTOCOL A, treatment for 12 days with undiluted THPF250523 induces more cell growth, supporting hair development than the other formulations used (p<0.05). However, the application of PROTOCOL A showed inferior results compared to PROTOCOL B (p<0.05). Finally, the activity of the WNT/beta-catenin system, which plays a central role in the generation of hair follicles during embryonic development and in the life cycle of follicles in the adult individual, has been characterised in parallel. Without this mechanism, there is no regrowth of hair, hair, eyelashes and eyebrows, but also the activation of melanogenesis, which leads to hair pigmentation [doi:10.3390/ijms21144915.]. The data show that following application of PROTOCOL A, WNT activity was more significant after stimulation with undiluted THPF250523, compared with the other formulations (p<0.05); again, it was found that although there was an increase in WNT activity following application of Protocol A compared with the control (p<0.05), this increase was less than with Protocol B (p<0.05). These results are directly proportional to the previous data described, indicating that undiluted THPF250523 supports and promotes compensatory mechanisms for the maintenance of the 3D organotypic system and the development of the hair bulb and subsequent hair growth. Furthermore, it can be stated that both stimulation protocols at the hair bulb level are effective with optimal results even after treatment discontinuation after day 8; however, a greater beneficial effect was found with PROTOCOL B.

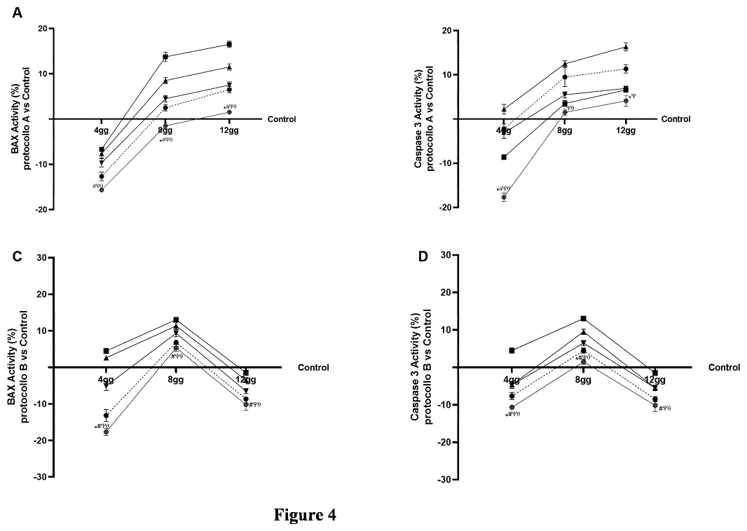

Finally, the role of BAX and Caspase 3, biological markers involved in the induction of cell death mechanisms, was described. As can be seen from FIG. 4, following application of Protocol A, BAX and Caspase 3 activity was strongly reduced after stimulation with the undiluted test product compared to the other concentrations tested (p<0.05). The data obtained were also confirmed following the application of PROTOCOL B, which proved to suppress apoptotic mechanisms, in a statistically significant manner (p<0.05), in the interval between day 8 and day 12 of treatment. These results correlate directly with previously observed data, indicating that undiluted THPF250523 supports and promotes compensatory mechanisms for the maintenance of the 3D organotypic system and the development of the hair bulb and subsequent hair growth. Furthermore, it can be stated that both stimulation protocols at the hair bulb level are effective with optimal results even after treatment discontinuation after day 8; however, a greater beneficial effect was found with PROTOCOL B.

The results obtained show that the formulation based on undiluted THPF250523, as opposed to the other formulations tested, provides the following effects:
positive effects of maintaining cell functionality and survival and consequently maintaining hair health.
positive effects limiting the onset of inflammation and oxidative stress at the level of the 3D organotypic hair bulb system set up and consequently limits cell death mechanisms.
beneficial effects following the application of both treatment protocols with optimal results also following the second treatment protocol after discontinuation of stimulation on day 8.

Therefore, THPF250523 represents a possible and viable alternative to prevent hair loss and to support the development and maintenance of the hair bulb after a 12-day treatment protocol.

Ex Vivo Evaluation of the Formulation 9 Diluted ¼ with 0.9% NaCl (Experimental Code THPF250523) on Scalp Hair Follicles Using Two Different Protocols of Administration

### Isolation and Culture of Scalp Hair Follicles

For the realisation of the ex-vivo model, portions of skin tissue extracted from the back of mice were cut into small pieces (4 mm×4 mm) and the skin fragments obtained were processed according to a standard protocol reported in the literature [Molina, B. et al., Microscopy Research, 2020:8, 9-30] to recreate ex-vivo the epithelial compartment surrounding the hair bulb. In more detail, newborn mice were sacrificed, kept in 70% alcohol for 10 minutes and washed twice for 5 minutes with PBS. Subsequently, the tissues were cut into small pieces (4 mm×4 mm) and the skin fragments were incubated in 0.25% dyspase II (Bacillus polymyxa, Gibco, BRL) in DMEM/F12 (1:1; Gibco-BRL, Thermo Fisher Scientific, Waltham, Massachusetts, USA) for 12-18 h at 4° C. After that, the tissues were first rinsed in saline and excess adipose tissue was removed. The hair follicles were gently extracted and harvested using a dissecting microscope. After two more rinses, the bulbs were transferred to Dulbecco's modified Eagle's medium (DMEM, Gibco, Thermo Scientific, Waltham, Massachusetts, USA), supplemented with 10% foetal bovine serum (FBS; Gibco, Thermo Scientific, Waltham, Massachusetts, USA), 10 ng/ml epidermal growth factor (Invitrogen), 5 g/ml hydrocortisone, 5 g/ml insulin (Sigma-Aldrich), 200 mmol/L L-glutamine (Gibco, Thermo Scientific, Waltham, Massachusetts, USA) and antibiotics (100 U/ml penicillin and 100 g/ml streptomycin). The culture was incubated at 37° C., 95% humidity and 5% CO₂.

### Experimental Protocol

Portions of skin tissue extracted from the back of mice were used to evaluate the beneficial action of the product in modulating FP growth. Therefore, an ex-vivo model was used to analyse the key mechanisms contributing to hair bulb death by studying oxidative stress activation, antioxidant mechanisms such as SOD, and inflammatory processes such as NFKb, TNFa and the main cytokines involved. Furthermore, the main mechanisms involved in apoptotic and cell survival processes, such as the activation of Cytochrome C and the involvement of ERK/MAPK were evaluated. Finally, the mechanisms involved in hair bulb regeneration, such as cell proliferation, cell cycle control proteins and the role of new substances in activating the WNT/beta-catenin pathway were studied.

Test MTT, the production of reactive oxygen species (ROS), the ELISA assay for TNFα, the ELISA assay for Interleukin 1β (IL-1β), the Assay for Wnt activity and the Assay for BAX activity were carried out using the same material and methods utilized for the in vitro tests.

### ELISA Assay for Superoxide Dismutase (SOD)

The SOD level was measured following the manufacturer's instructions (Cayman's Superoxide Dismutase Assay Kit), which detects all three types of SOD (Cu/Zn, Mn and FeSOD) [13]. Briefly, the level of SOD present in cell lysates was measured by comparing the data with a standard curve (0.05-0.005 U/mL). Total cell lysates were obtained with cold PBS1x. The absorbance of all samples was measured with a spectrometer (Infinite 200 Pro MPlex, Tecan, Männedorf, Switzerland) at 480 nm and the results are expressed as mean (%) compared to the control. The concentration is expressed in ng/ml relative to a standard curve (range 0-0.005 U/mL) and the results are expressed as a percentage (%) relative to the control (line 0).

### ELISA Assay for NFKB

The DNA-binding activity of NFKB was analyzed by an enzyme-linked immunosorbent assay (ELISA), following the manufacturer's instructions (Cayman Chemical Company, Ann Arbor, MI, USA). Nuclear extracts were prepared using a nuclear extraction protocol [Uberti, F. et al., J. Ovarian Res. 2017, 10, 61.] and the NFKB contained in these extracts was detected with the addition of a specific primary antibody. An HRP-conjugated secondary antibody was added to provide sensitive colorimetry measured by a spectrometer (Infinite 200 Pro MPlex, Tecan, Männedorf, Switzerland) at 450 nm and the concentration was calculated by comparing the results with the standard curve. The concentration is expressed in ng/mL relative to a standard curve standard curve (range 0.05-0.005 U/ml) and the results are expressed as a percentage relative to the control (line 0).

### Assay for Cytochrome C Activity

The Cytochrome-C ELISA kit (MyBiosource, San Diego, CA, USA) was used to measure the amount of cytochrome C in cell lysates, following the manufacturer's instructions. Briefly, 100 μL of each sample was added and incubated at 37° C. for 90 minutes; then, the material was removed and 100 μL of detection solution A was added and incubated for 45 minutes at 37° C. At the end of the incubation time, the wells were washed and 100 μL of detection solution B was added to each well and then incubated for 45 min at 37° C. Then 90 μL of substrate solution was added to each well and the plate was incubated for 20 min at 37° C. in the dark. 50 μL of stop solution was used to stop the reaction and the absorbance was analysed with a spectrometer (Infinite 200 Pro MPlex, Tecan, Männedorf, Switzerland) at 450 nm and the concentration was expressed in ng/ml by comparing the data with the standard curve (range 15.6 nmol/L-500 nmol/L).

### Assay for ERK/MAPK Activity

Analysis of ERK/MAPK activity was performed using the InstantOne™ ELISA (Thermo Fisher, Milan, Italy) on chondrocyte lysates [Molinari C et al., Oxid Med Cell Longev.; 2019:2843121]. Cells at the end of the treatments were lysed with 100 μL of cell lysis buffer and 50 μL/well of each sample was tested in InstantOne ELISA microplates; the antibody cocktail was added to each well and incubated for 1 hour at room temperature under agitation. At the end of the time, the detection reagent was added to each well and, after 20 minutes, the reaction was stopped by adding the stop solution. The absorbance was measured with a spectrometer (Infinite 200 Pro MPlex, Tecan, Männedorf, Switzerland) at 450 nm. The results were expressed as mean absorbance (%) compared to the control. The concentration is expressed in ng/mL relative to a standard curve (range 62.5-2000 μg/mL) and the results are expressed as a percentage relative to the control (line 0).

### ELISA Assay for CDK1

The ELISA kit for CDK1 (MyBiosource, San Diego, CA, USA) was used according to the manufacturer's instructions [doi:10.1016/j.stemcr.2015.01.019]. HOSEpiC cells were lysed with cold 1× Phosphate-Buffered Saline (PBS, Merck Life Science, Rome, Italy), centrifuged at 5000×g for 5 min and 100 μL of each sample was analysed by reading the sample absorbance at 450 nm with a spectrometer (Infinite 200 Pro MPlex, Tecan, Männedorf, Switzerland). The results were obtained by comparing the data with the standard curve (ranging from 0.156 to 10 ng/mL) and were expressed as a percentage compared to the control (line 0) of five independent experiments performed in triplicate.

### Results

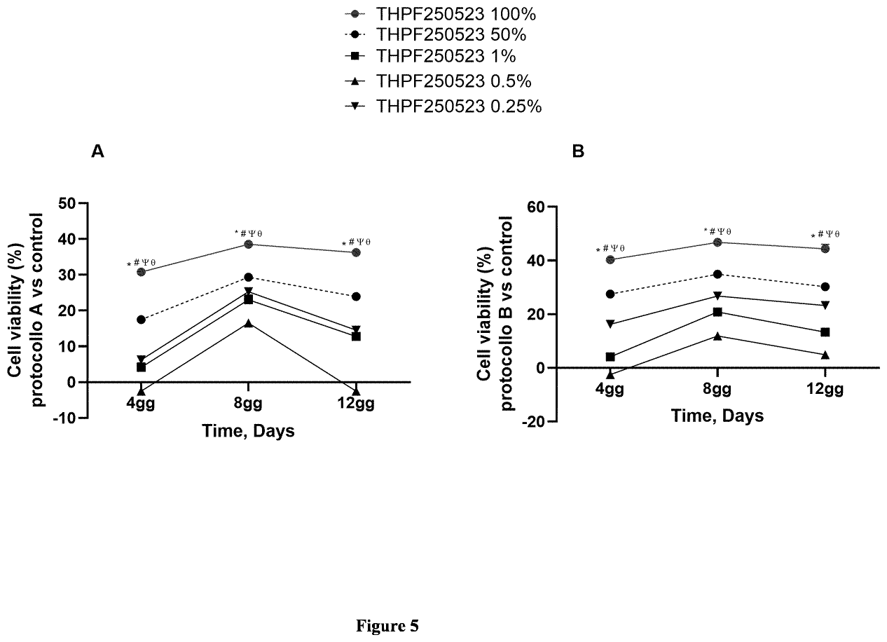

FIGS. 5/A and 5/B, after application of PROTOCOL A, show that cell viability was statistically significantly higher after stimulation with the undiluted test product (100%) than with the other concentrations tested (p<0.05). The data obtained were also confirmed after the application of PROTOCOL B (FIG. 5/B), which improved cell viability in a statistically significant manner compared to the other concentrations tested (p<0.05), in the interval between day 8 and day 12 of treatment. This confirmed the results obtained after application of both protocols on an in vitro model; in fact, even on an ex-vivo model, PROTOCOL B showed better results in maintaining cell viability and functionality (p<0.05), which did not alter after discontinuation of stimulation after the eighth day of treatment.

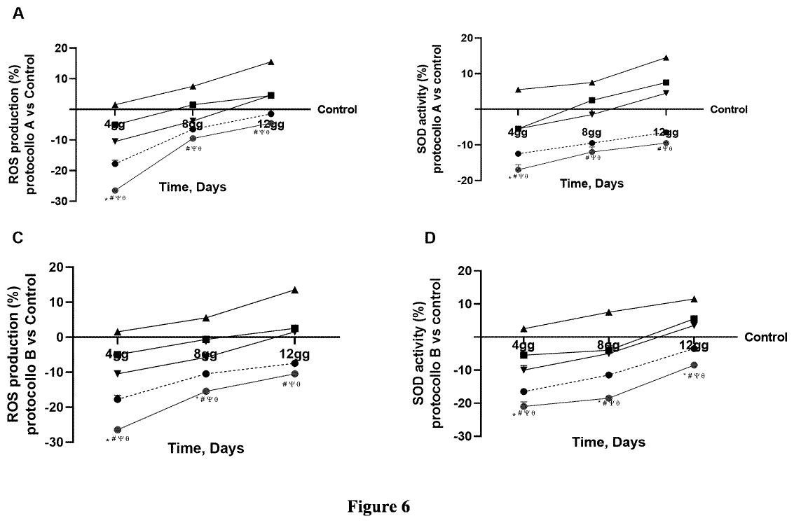

With regard to oxidative stress, FIG. 6 further demonstrates that ROS production was drastically reduced after treatment with undiluted THPF250523, consistent with what was defined by cell viability (p<0.05). PROTOCOL B (FIG. 6/D) demonstrated lower ROS values after day 8 of treatment. These data are further confirmed by determining the levels of SOD, which limits oxidative stress-induced cell damage, preventing functionality and limiting cellular ageing [Prie B E, et al., J Med Life. 2016; 9(1): 79-83]. SOD activity for both protocols was lower after treatment with undiluted THPF250523, in a statistically significant manner compared to the other concentrations examined (p<0.05). The data confirm the results obtained in the in vitro model, demonstrating that undiluted THPF250523 appears to have strong potential for maintaining cell function and survival, showing potential as an anti-hair loss agent.

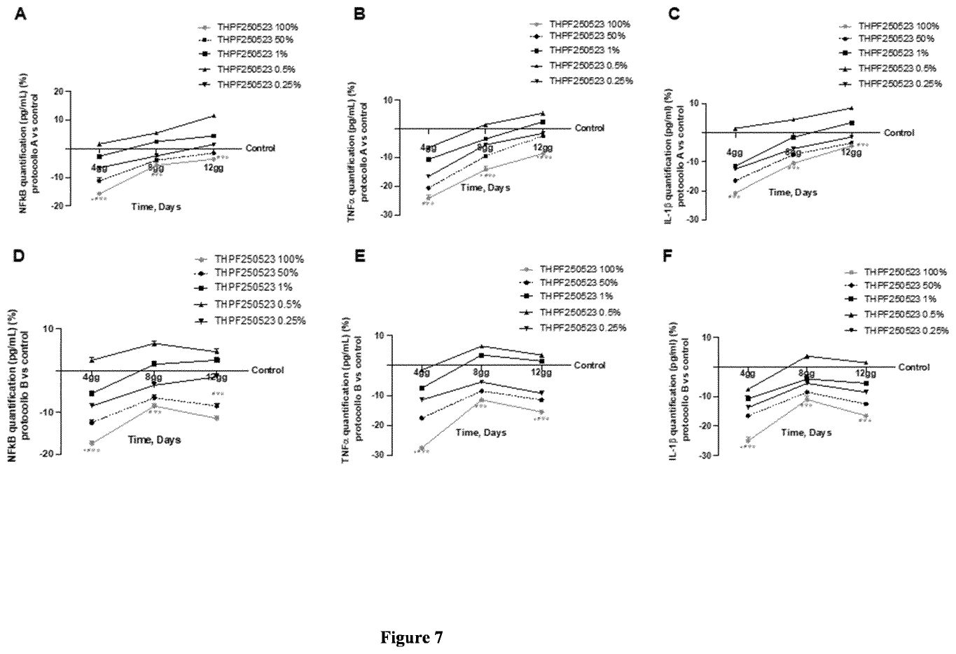

Subsequently, the main biological markers involved in triggering the inflammatory process were also investigated in ex-vivo models, in order to obtain further confirmation of the positive effects promoted by THPF250523 on hair bulb health. The data shown in FIG. 7 demonstrate that THPF250523 at 100% promotes the maintenance of reduced levels of TNFα, NFkB and IL-1β during the treatment period of PROTOCOL A compared to the other formulations (p<0.05); in fact, the data demonstrate a good preventive and protective action on the inflammatory mechanism, confirming the results observed on the in vitro model. Consistently, the application of PROTOCOL B (FIG. 7/D-F) also confirmed previous result, with statistically significant improvements over PROTOCOL A (p<0.05).

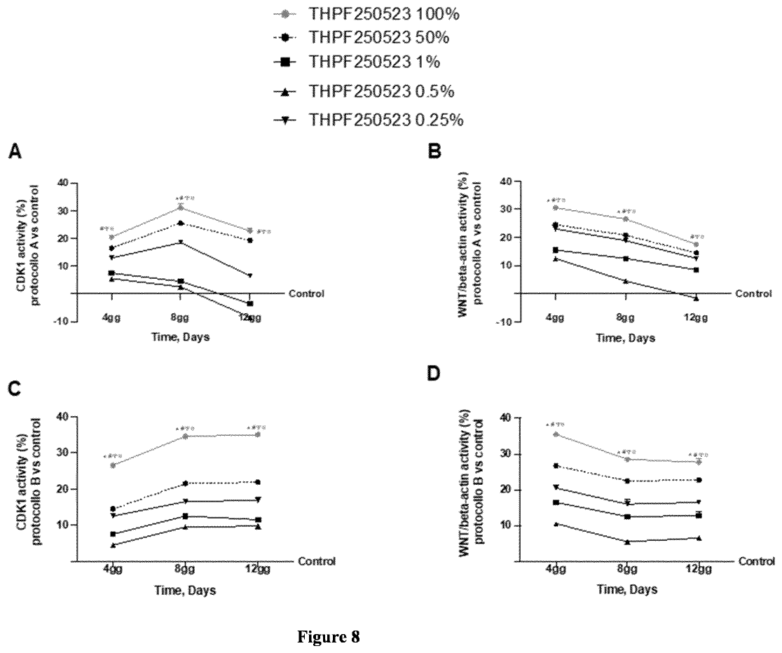

Consistent with what was studied on the in vitro model, analysis of the mechanisms involved with cell growth was crucial in order to describe the functional status and maintenance of hair growth by THPF250523. On the ex-vivo model, CDK1 activity (FIGS. 8/A and C) was also increased following the application of PROTOCOL A (p<0.05); in fact, as on the in-vivo model, treatment for 12 days with uniluted THPF250523 induced more cell growth, supporting hair growth than the other formulations used (p<0.05). The application of PROTOCOL B showed greater results than PROTOCOL A (p<0.05), with an increase in CDK1 activity in the interval between days 8 and 12, following treatment discontinuation after day 8. Finally, the activity of the WNT/β-catenin system was characterised in parallel. The data show that following application of PROTOCOL A, WNT activity was better after stimulation with undiluted THPF250523, compared to the other formulations (p<0.05); also in this case, it was found that following application of PROTOCOL B, the increase in WNT activity was higher than with PROTOCOL A (p<0.05) (FIGS. 8/B and D). These results are directly proportional to previously observed data, indicating that undiluted THPF250523 supports and promotes compensatory mechanisms for the maintenance of hair bulb development and subsequent hair growth even in an ex-vivo model. Both stimulation protocols at the hair bulb level are effective with optimal results even after treatment discontinuation after day 8. Better performance data were found following the use of PROTOCOL B.

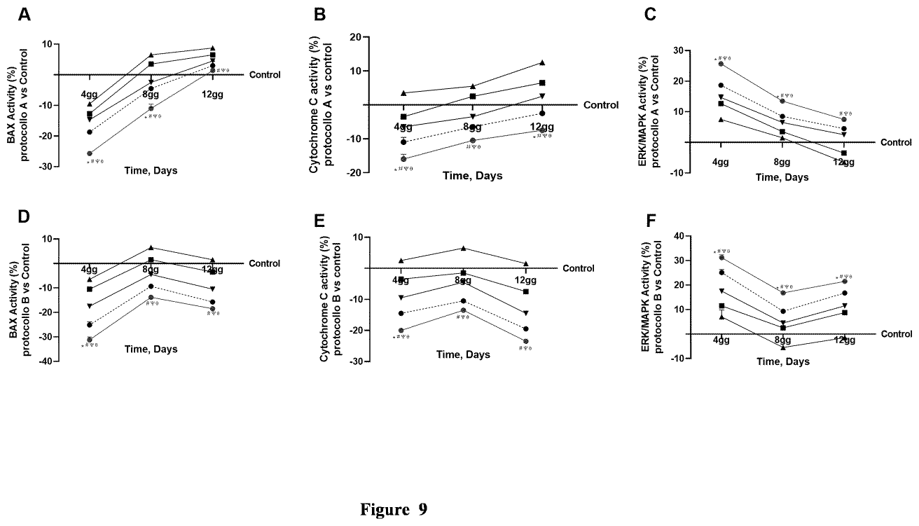

Finally, the role of Cytochrome C, a biological marker involved in the induction of programmed cell death mechanisms, was described (FIG. 9). The initiation of the apoptotic mechanism is directly correlated with the activation of BAX, which stimulates the release of cytochrome-C from the mitochondria, acting as a downstream cell death signal

The results obtained show that undiluted THPF250523, as opposed to diluted formulations, provides beneficial effects, maintaining cell function and survival, and consequently maintaining hair health.

Undiluted THPF250523 also counteracts the onset of inflammation and oxidative stress in the ex-vivo model of the hair bulb set up and consequently limiting cell death mechanisms. The beneficial effects of undiluted THPF250523 following the application of both treatment protocols were confirmed also following the second treatment protocol after stopping stimulation on day 8.

1. A method for preventing hair loss or improving hair growth in subjects in need thereof, said method comprising:
administering to said subjects topical formulations containing peroxyacetic or peroxyhalogenated acetic acids or a combination of acetic acid or halogenated acids with a hydrogen peroxide solution in admixture with suitable carriers, vehicles or complementary useful ingredients.

2. The method according to claim 1 wherein the peroxyacetic or peroxyhalogenated acetic acids are selected from peroxytrichloroacetic acid, peroxydichloroacetic acid, peroxymonochloroacetic acid or peracetic acid.

3. The method, according to claim 1, wherein said topical formulations contains trichloroacetic acid, dichloroacetic acid, monochloroacetic acid or acetic acid and 28-30% hydrogen peroxide.

4. The method according to claim 3, wherein the weight ratio of acetic or halogenated acetic acids to hydrogen peroxide 28-30% is between 22.0 and 5.3.

5. The method according to claim 3, wherein said topical formulations contains trichloroacetic acid, dichloroacetic acid, monochloroacetic acid or acetic acid in a range comprised between 33.0 and 0.08% weight.

6. The method according to claim 3 in form of kit containing separate administration forms for the hydrogen peroxide solution and for the acetic or halogenated acetic acids.

7. The method according to claim 2, wherein said topical formulations contains peroxytrichloroacetic acid, peroxydichloroacetic acid or peroxymonochloroacetic acid or peracetic acid in a range comprised between 2.0 and 0.8% by weight.

8. The method according to claim 1, wherein the carriers, vehicles or complementary useful ingredients are selected from glycerol in concentration by weight 2.0-10.0%, water or a 0.9% NaCl water solution added individually in amount comprised between 52-94% by weight, 30% aqueous ammonia in a concentration by weight ranging from 12.0 to 0.03%.

9. The method according to claim 1, wherein said topical formulations have a pH from 2 to 3.

10. A cosmetic, aesthetic or therapeutic method of preventing hair loss or improving hair growth comprising
topically applying on the skin of a subject in need thereof of an effective amount of the compositions of claim 1.

11. The method according to claim 10 wherein the compositions are topically administered at T0 and continued every other day for a total of 12 days of treatment.

12. The method according to claim 10 wherein the compositions are topically administered at T0 and continued every other day for 8 days of treatment.

13. The method according to claim 11, wherein the compositions are diluted before administration to 25%, 1.25% and to 0.25% weight/volume with demineralized water or physiological 0.9% NaCl/demineralized water.
